(19) United States
(12) Patent Application Publication
(10) Pub. No.: US 2022/0134388 A1
(43) Pub. Date: May 5, 2022

(54) CLEANING DEVICE

(71) Applicant: SOPHONIX CO., LTD., Beijing (CN)

(72) Inventors: Shiliang ZHOU, Beijing (CN); Kuiliang HAN, Beijing (CN); Xiqiang ZHANG, Beijing (CN); Yongcheng SUN, Beijing (CN); Haiyang ZHANG, Beijing (CN)

(73) Assignee: SOPHONIX CO., LTD., Beijing (CN)

(21) Appl. No.: 17/578,456

(22) Filed: Jan. 19, 2022

Related U.S. Application Data
(63) Continuation of application No. PCT/CN2020/104910, filed on Jul. 27, 2020.

(30) Foreign Application Priority Data
Mar. 30, 2020 (CN) 202010235541.8

(51) Int. Cl. B08B 1/00

(52) U.S. Cl. CPC B08B 1/008

(57) ABSTRACT

The present application relates to a cleaning device, including an inverted U-shaped frame body, which includes a horizontal beam, and both ends of the beam are respectively connected with vertical beams perpendicular to the beam; a horizontal bearing plate for bearing a cleaning mechanism and a first driving mechanism for driving the bearing plate to move up and down are provided in the frame body; the cleaning mechanism comprises a magnetic sleeve assembly and a magnetic rod in a sleeve connection with it, and the magnetic rod is connected with a second driving mechanism for driving it to move up and down relative to the magnetic sleeve assembly; and movements between the first driving mechanism and the second driving mechanism are coordinated by a PLC.

### CROSS-REFERENCE TO RELATED APPLICATION

This application is a continuation of PCT application serial no. PCT/CN2020/104910 filed on Jul. 27, 2020, which claims the priority and benefit of China patent application no. 202010235541.8 filed on Mar. 30, 2020. The entirety of each of the above-mentioned patent applications is hereby incorporated by reference herein and made a part of this specification.

### TECHNICAL FIELD

The present application relates to a technical field of a medical equipment, and in particular to a cleaning device.

### BACKGROUND ART

Luminescence immunoanalyzer combines micro magnetic bead solid-phase carrier platform with chemiluminescence testing technology. It not only has characteristics of high sensitivity, accurate results and automation of magnetic portionicle chemiluminescence testing, but also has the advantages of fast testing, simple operation and portability. Therefore, it is especially suitable for a POCT (point of care-testing) application, such as a rapid testing of an outpatient service and family self-testing, etc.

A micro magnetic bead is a carrier for conducting the test solution analysis, which needs to be cleaned before the final luminescence data is tested and read accurately through the testing and reading module. Therefore, the cleaning of the micro magnetic bead is a necessary guarantee for accuracy of analytical data.

The existing method for cleaning the micro magnetic bead has low degree of automation, cumbersome operation, low efficiency and poor cleaning effect, thus it cannot meet a demand of modern testing.

### SUMMARY

An object of the present application is to provide a cleaning device, which has a high degree of automation, stable operation, good cleaning effect and high clean efficiency.

The above object of the present application is realized by a following technical solution.

A cleaning device includes an inverted U-shaped frame body, which includes a horizontal beam, and both ends of the beam are respectively connected with vertical beams perpendicular thereto; a horizontal bearing plate for bearing a cleaning mechanism and a first driving mechanism for driving the bearing plate to move up and down are provided in the frame body; the cleaning mechanism includes a magnetic sleeve assembly and a magnetic rod in a sleeve connection with it, and an upper end of the magnetic rod is connected with a second driving mechanism for driving it to move up and down relative to the magnetic sleeve assembly;

the magnetic sleeve assembly includes a magnetic sleeve seat, a magnetic sleeve and the magnetic rod; the bearing plate is provided with multiple vertical through holes, of which a lower end is fixedly communicated with the hollow tubular magnetic sleeve seat, and the lower end of the magnetic sleeve seat is in the sleeve connection with the tubular magnetic sleeve seat with an upper opening; and the magnetic rod passes through the through hole and the magnetic sleeve seat downward respectively, and is in the sleeve connection with the magnetic sleeve;

the first driving mechanism includes a first plate, which is provided at a rear side of and fixedly connected with the bearing plate; a horizontal supporting plate fixedly connected with two vertical beams, is provided below the first plate; a vertically provided first motor is fixedly connected with an upper end of the beam, of which an output end penetrates through the beam and is connected with a vertical first lead screw; and the first plate is fixed connected with a first lead screw nut adapted to the first lead screw, and further provided with a first guiding mechanism to guide it to move up and down;

the second driving mechanism includes a second plate provided above the first plate, and the rear side of the first plate is fixedly connected with a vertical motor frame, of which the upper end is fixedly provided with a vertical second motor; an output end of the second motor is connected with a vertical second lead screw, and the second plate is fixedly connected with a second lead screw nut adapted to the second lead screw, and further provided with a second guiding mechanism for guiding it to move up and down; and the front side of the second plate is fixedly connected with a clamping plate for clamping the magnetic rod; and

the first motor and the second motor are connected with a PLC coordinating their movement.

In the above technical solution, the first motor can drive the first plate, the rearing plate, the clamping plate and the second plate to move up and down together via the first lead screw. When the first motor rotates to drive the first lead screw to drive the first plate and the second plate to move downward together, first, the magnetic sleeve seat is sleeved on the magnetic sleeve in the reagent strip. Then, the second motor rotates to drive the second plate to move up and down via the second lead screw, and the clamping plate fixedly connected on the second plate drives the magnetic rod to move up and down, so that the lower end of the magnetic rod moves away from or close to a bottom of the magnetic sleeve. The magnetic rod abuts against or moves away from the bottom of the magnetic sleeve, so that a micro magnetic bead in a cleaning hole on the reagent strip is attracted by or separated from the magnetic rod, so as to achieve the purpose of cleaning the micro magnetic bead. Working states of the first motor and the second motor are coordinated and controlled by the PLC.

In some embodiments, the magnetic rod is rod-shaped, of which a lower portion is a magnetic portion, and an upper portion is provided with a retaining ring protruding outward and a top end is provided with a vertical internal threaded hole.

In the above technical solution, the magnetic portion at the lower part of the magnetic rod can attract the micro magnetic bead, and the magnetic rod can be clamped and fixed with the clamping plate via the retaining ring and the internal thread, so that the magnetic rod can move up and down with the clamping plate.

In some embodiments, the clamping plate is provided with multiple stepped grooves with openings facing the second plate; a step at the lower end of the stepped groove is provided with a clamping hole adapted to the magnetic rod; the upper end of the magnetic rod extends through the clamping hole upward and the retaining ring abuts against the lower end of the clamping plate, and a fastener is threaded into the internal threaded hole.

In the above technical solution, the retaining ring of the magnetic rod can abut against the lower end of the clamping plate, and the upper end of the magnetic rod is fixed at the upper end of the step of the stepped groove by the fastener, so that the magnetic rod can be firmly connected with the clamping plate, and the magnetic rod can move synchronously with the clamping plate.

In some embodiments, the first guiding mechanism includes first sliding holes provided on the first plate and at two sides of the first lead screw, and each vertical first sliding rod extends through each first sliding hole, and both ends of each sliding rod are fixedly connected with the beam and the supporting plate respectively; the second guiding mechanism includes second sliding holes provided on the second plate and at two sides of the second lead screw, and each vertical second sliding rod extends through each second sliding hole, and both ends of each sliding rod are fixedly connected with the beam and the supporting plate respectively.

In the above technical solution, the first plate can move up and down under the guidance of the first sliding rods at its both sides, and the second plate can move up and down under the guidance of the second sliding rods at both sides, so that stability of the up and down movements of the first plate and the second plate can be ensured.

In some embodiments, a first sliding sleeve is coaxially fixedly provided in the first sliding hole, and the first sliding sleeve is slidably connected with the first sliding rod; a second sliding sleeve is coaxially fixedly provided in the second sliding hole, and the second sliding sleeve is slidably connected with the second sliding rod.

In the above technical solution, the first sliding rod can slide in the first sliding sleeve, which reduce friction and improve the stability of the up and down movement of the first plate. The second sliding rod can slide in the second sliding sleeve, which also reduce the friction and improve the stability of the up and down movement of the second plate.

In some embodiments, the other end of the first lead screw and the other end of the second lead screw are both free ends.

In the above technical solution, the other end of the first lead screw and the other end of the second lead screw are both free ends, which can reduce a volume of the device, facilitate installation and maintenance and improve reliability of a device operation.

In some embodiments, the first plate is provided with pocket holes for the second sliding rod and the second lead screw respectively; the second plate is provided with the pocket holes for the first sliding rod and the first lead screw respectively.

In the above technical solution, by providing the pocket holes, the smooth installation of the first sliding rod, the second sliding rod, the first lead screw and the second lead screw can be ensured, and there is no interference with the first plate and the second plate, so as to ensure that the first plate and the second plate can move up and down normally.

In some embodiments, numbers of the through holes, the cleaning mechanisms and the stepped grooves on the bearing plate are eight.

In the above technical solution, by providing eight cleaning mechanisms on the bearing plate, during the process of the first motor driving the first plate and the second plate to move up and down synchronously, eight cleaning mechanisms can move up and down at the same time, so that the micro magnetic beads in eight reagent strips can be cleaned at the same time, which greatly improves the cleaning efficiency and productivity.

In summary, the beneficial technical effects of the present application are as follows.

1. By providing the first driving mechanism and the second driving mechanism, not only the cleaning mechanism moving up and down synchronously with the first plate and the second plate, but also the magnetic rod moving close to or away from the magnetic sleeve by the second driving mechanism can be achieved. Therefore, the lower end of the outer side of the magnetic sleeve can adsorb the micro magnetic beads, and the micro magnetic beads at the lower end of the outer side of the magnetic sleeve also can be separated from the magnetic sleeve, so as to achieve the purpose of cleaning the micro magnetic beads. The device has high degree of automation, stable operation, good cleaning effect and high cleaning efficiency.

2. By providing the first guiding mechanism and the second guiding mechanism, accuracy of the up and down movements of the first plate and the second plate is high, and the operation is stable.

3. By providing eight through holes, cleaning mechanisms and stepped grooves on the bearing plate, the micro magnetic beads in eight reagent strips can be cleaned at the same time, which greatly improves the cleaning efficiency and productivity.

### BRIEF DESCRIPTION OF THE DRAWINGS

FIG. 1 is a front view structural diagram of the present application;

FIG. 2 is a structural diagram of the present application without a clamping plate;

FIG. 3 is a back view structural diagram of the present application;

FIG. 4 is the structural diagram of the clamping plate in the present application;

FIG. 5 is an exploded diagram of a cleaning mechanism in the present application;

FIG. 6 is the structural diagram of a reagent strip rack and a reagent strip in the present application.

### DETAILED DESCRIPTION

The present application will be described in further detail below in combination with the drawings.

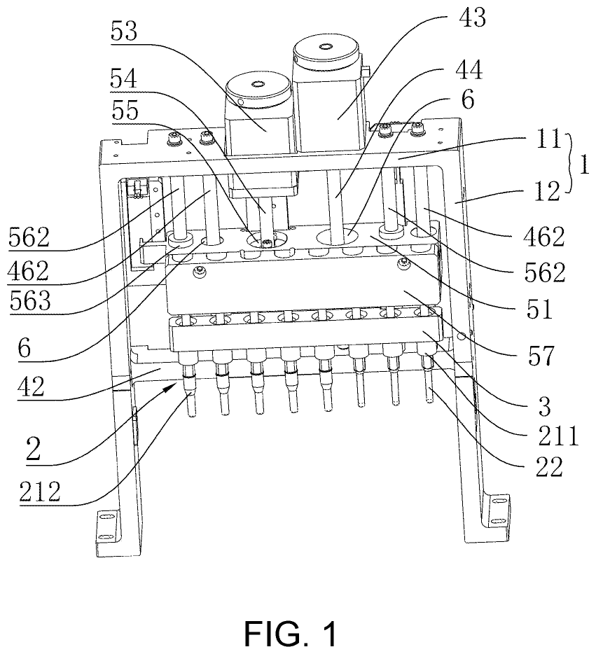

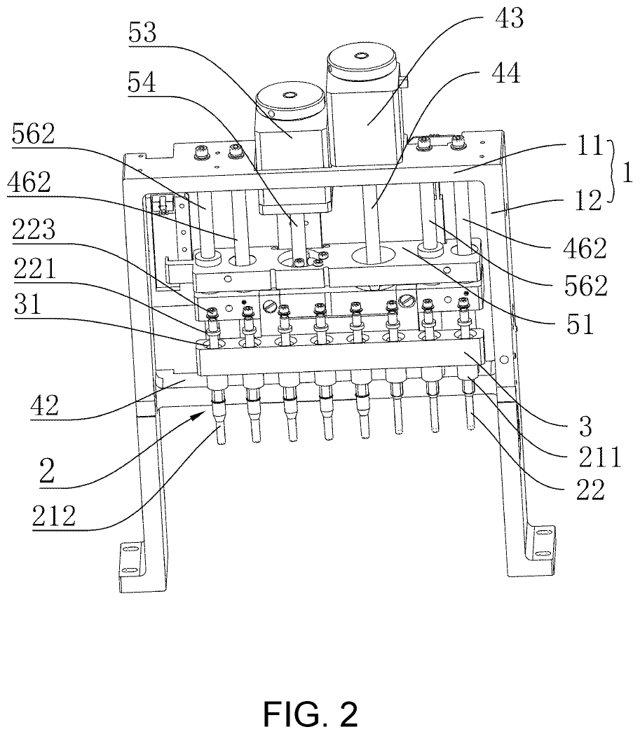

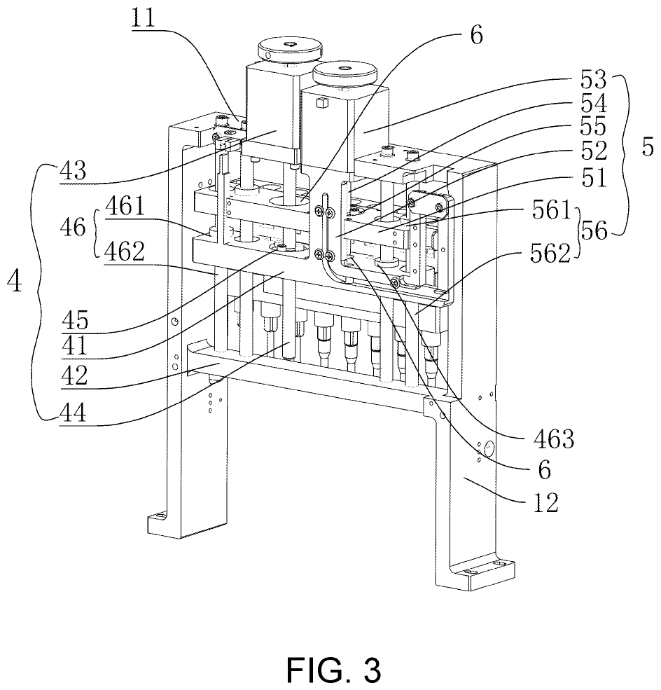

As shown in FIGS. 1-3, a cleaning device includes an inverted U-shaped frame body 1, which includes a horizontal beam 11, and both ends of the beam 11 are respectively connected with vertical beams 12 perpendicular to it. A horizontal bearing plate 3 for bearing a cleaning mechanism 2 and a first driving mechanism 4 for driving the bearing plate 3 to move up and down are provided in the frame body 1. The cleaning mechanism 2 includes a magnetic sleeve assembly 21 and a magnetic rod 22 in a sleeve connection with it, and an upper end of the magnetic rod 22 is connected with a second driving mechanism 5 for driving it to move up and down relative to the magnetic sleeve assembly 21.

The magnetic sleeve assembly 21 includes a magnetic sleeve seat 211, a magnetic sleeve 212 and the magnetic rod 22. The bearing plate 3 is provided with multiple vertical through holes 31 of which a lower end is fixedly communicated with the hollow tubular magnetic sleeve seat 211, and the lower end of the magnetic sleeve seat 211 is in the sleeve connection with the tubular magnetic sleeve seat 211 with an upper opening. The magnetic rod 22 extends through the through hole 31 and the magnetic sleeve seat 211 downward respectively, and is sleeved in the magnetic sleeve 212.

The first driving mechanism 4 includes a first plate 41, which is provided at a rear side of and fixedly connected with the bearing plate 3. A horizontal supporting plate 42 fixedly connected with two vertical beams 12 is provided below the first plate 41. A vertically provided first motor 43 is fixedly connected with an upper end of the beam 11, of which an output end penetrates through the beam 11 and is connected with a vertical first lead screw 44. The first plate 41 is fixedly connected with a first lead screw nut 45 adapted to the first lead screw 44, and is also provided with a first guiding mechanism 46 to guide it to move up and down.

The second driving mechanism 5 includes a second plate 51 provided above the first plate 41, and the rear side of the first plate 41 is fixedly connected with a vertical motor frame 52, of which the upper end is fixedly provided with a vertical second motor 53. An output end of the second motor 53 is connected with a vertical second lead screw 54, and the second plate 51 is fixedly connected with a second lead screw nut 55 adapted to the second lead screw 54, and is further provided with a second guiding mechanism 56 for guiding it to move up and down. The front side of the second plate 51 is fixedly connected with a clamping plate 57 for clamping the magnetic rod 22.

The forward or reverse rotation and operating time of the first motor 43 and the second motor 53 are coordinated and controlled by a PLC.

In an embodiment, in order to improve accuracy and stability of movements of the first plate 41 and second plate 51, both of the first lead screw 44 and the second lead screw 54 are T-shaped screws.

The first motor 43 can drive the first plate 41, the bearing plate 3, the clamping plate 57 and the second plate 51 to move up and down together via the first lead screw 44. When the first motor 43 rotates to drive the first plate 41 and the second plate 51 to move downward synchronously via the first lead screw 44, the magnetic sleeve seat 211 is firstly sleeved on the magnetic sleeve 212 in a reagent strip 8. Then, the second motor 53 rotates to drive the second plate 51 to move up and down via the second lead screw 54, and the clamping plate 57 fixedly connected on the second plate 41 drives the magnetic rod 22 to move up and down, so that the lower end of the magnetic rod 22 moves away from or close to a bottom of the magnetic sleeve 212. The magnetic rod 22 abuts against or moves away from the bottom of the magnetic sleeve 212, so that a micro magnetic bead in a cleaning hole is attracted by or separated from the magnetic rod 22, so as to achieve the purpose of cleaning the micro magnetic bead. Working states of the first motor 43 and the second motor 53 are coordinated and controlled by a PLC.

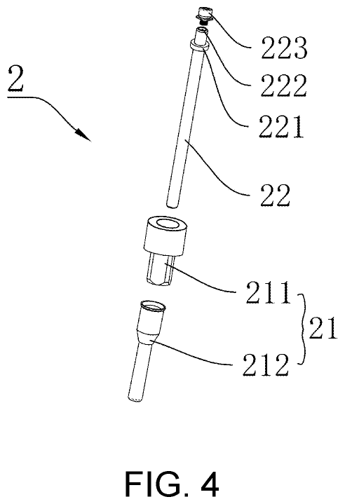

As shown in FIG. 4, the magnetic rod 22 is rod-shaped, its lower portion is a magnetic portion, which can attract the micro magnetic bead. An upper portion of the magnetic rod 22 is provided with a retaining ring 221 protruding outward, and a top end of the magnetic rod 22 is provided with a vertical internal threaded hole 222. The magnetic rod 22 can be clamped and fixed with the clamping plate 57 by providing the retaining ring 221 and the internal thread, so that the magnetic rod 22 can move up and down with the clamping plate 57.

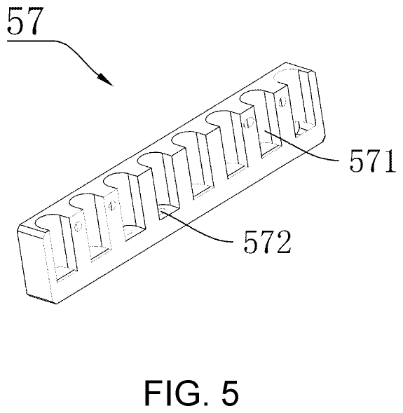

As shown in FIG. 5, the clamping plate 57 is provided with multiple stepped grooves 571 with openings facing the second plate 51. A step at the lower end of the stepped groove 571 is provided with a clamping hole 572 adapted to the magnetic rod 22. The upper end of the magnetic rod 22 extends through the clamping hole 572 upward and the retaining ring 221 abuts against the lower end of the clamping plate 57, and a fastener 223 is threaded into the internal threaded hole 222. The upper end of the magnetic rod 22 is fixed at the upper end of the stepped groove 571 by the fastener 223, so that the magnetic rod 22 can be firmly connected with the clamping plate 57, and the magnetic rod 22 can move up and down synchronously with the clamping plate 57.

The first guiding mechanism 46 In an embodiment includes first sliding holes 461 provided on the first plate 41 and at two sides of the first lead screw 44, and each vertical first sliding rod 462 extends through each first sliding hole 461, and both ends of each sliding rod 462 are fixedly connected with the beam 11 and the supporting plate 42 respectively. The second guiding mechanism 56 includes second sliding holes 561 provided on the second plate 51 and at two sides of the second lead screw 54, and each vertical second sliding rod 562 extends through each second sliding hole 561, and both ends of each sliding rod 562 are fixedly connected with the beam 11 and the supporting plate 42 respectively. The first plate 41 can move up and down under the guidance of the first sliding rods 462 at both sides, and the second plate 51 can move up and down under the guidance of the second sliding rods 562 at both sides, so that stability of up and down movements of the first plate 41 and the second plate 51 can be ensured.

In order to reduce friction and make the movement between the plate and the sliding rod more stable and smooth, a first sliding sleeve 463 is coaxially fixedly provided in the first sliding hole 461, and the first sliding sleeve 463 is slidably connected with the first sliding rod 462. A second sliding sleeve 563 is coaxially fixedly provided in the second sliding hole 561, and the second sliding sleeve 563 is slidably connected with the second sliding rod 562.

In an embodiment, the other end of the first lead screw 44 and the other end of the second lead screw 54 are both free ends, so as to reduce a volume of the device, facilitate installation and maintenance and improve operation reliability of a device.

In order to ensure the smooth installation of the first sliding rod 462, the second sliding rod 562, the first lead screw 44 and the second lead screw 54 and ensure that there is no interference between the first plate 41 and the second plate 51, the first plate 41 is provided with pocket holes 6 for the second sliding rod 562 and the second lead screw 54 respectively, and the second plate 51 is provided with the pocket holes 6 for the first sliding rod 462 and the first lead screw 44 respectively.

In an embodiment, numbers of the through holes 31, the cleaning mechanisms 2 and the stepped grooves 571 on the bearing plate 3 are eight. By providing eight cleaning mechanisms 2 on the bearing plate 3, the first motor 43 can drive eight cleaning mechanisms 2 to move up and down with the first plate 41 and the second plate 51, and the second motor 53 drives eight magnetic rod 22 to move up and down at the same time relative to the magnetic sleeve 212. Eight cleaning mechanisms 2 can work at the same time, so that the micro magnetic beads in eight reagent strips 8 can be cleaned at the same time, which greatly improves the cleaning efficiency and productivity.

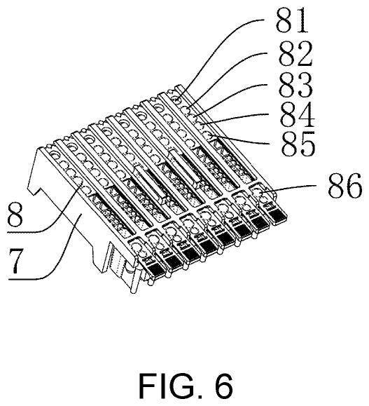

FIG. 6 is the structure diagram of a reagent strip rack 7 with eight reagent strips 8.

Referring to FIGS. 1, 2 and 6, the working process of the present application is as follows.

First, the magnetic sleeve seat 211 is sleeved on the magnetic sleeve 212. The reagent strip rack 7 is placed under the frame body 1 at first, and the magnetic sleeve placing hole 86 on the reagent strip 8 is placed just below the magnetic sleeve seat 211. The magnetic rod 22 is raised by the second motor 53, and then the second motor 53 stops working, and the first motor 43 rotates forward. The first lead screw 44 drives the first plate 41, the bearing plate 3, the clamping plate 57 and the second plate 51 to move downward synchronously, so that the magnetic sleeve seat 211 is sleeved on the magnetic sleeve 212 in the magnetic sleeve placing hole 86 on the reagent strip 8.

Then, the first motor 43 rotates reversely to drive the first plate 41, the bearing plate 3, the clamping plate 57 and the second plate 51 to move upward synchronously via the first lead screw 44. The reagent strip rack 7 is moved, so that the reaction hole 81 on the reagent strip 8 is placed just below the magnetic sleeve 212. The first motor 43 rotates forward again to drive the first plate 41, the bearing plate 3, the clamping plate 57 and the second plate 51 to move downward synchronously via the first lead screw 44, so that the magnetic sleeve 212 is immersed in reaction solution in the reaction hole 81. The second motor 53 rotates forward to drive the first plate 41, the clamping plate 57 and the magnetic rod 22 to move downward by the second lead screw 54, so that the magnetic rod 22 abutted against the lower end of the magnetic sleeve 212. Since the lower portion of the magnetic rod 22 is magnetic, the micro magnetic beads in the reaction solution in the reaction hole 81 are adsorbed on an outer wall at the lower end of the magnetic sleeve 212 by the magnetic rod 22.

The first motor 43 rotates reversely to drive the first plate 41, the rearing plate 3, the clamping plate 57 and the second plate 51 to move upward synchronously via the first lead screw 44. The reagent strip rack 7 is moved, so that a first cleaning hole 82 on the reagent strip 8 is placed just below the magnetic sleeve 212. The first motor 43 rotates forward to drive the first plate 41, the rearing plate 3, the clamping plate 57 and the second plate 51 to move downward synchronously via the first lead screw 44. After the magnetic sleeve 212 adsorbed with the micro magnetic beads is immersed in the first cleaning hole 82, the second motor 53 rotates reversely to drive the first plate 41, the clamping plate 57 and the magnetic rod 22 to move upward via the second lead screw 54, so that the magnetic rod 22 moves away from the lower end of the magnetic sleeve 212. Due to the blocking of the magnetic sleeve 212, the micro magnetic beads originally adsorbed to the lower end of an outer side of the magnetic sleeve 212 lose the adsorption force of the magnetic rod 22. Under action of gravity, some of the micro magnetic beads will fall off into cleaning solution in the first cleaning hole 82. At this time, the forward rotation and reverse rotation of the first motor 43 are carried out alternately, which drives the magnetic sleeve 212 to move into and out of the cleaning solution in the first cleaning hole 82. Therefore, the magnetic sleeve 212 is immersed in and moved out of the cleaning solution in the first first cleaning hole 82. After several times of the above mixing process, the micro magnetic beads originally adsorbed on the magnetic sleeve 212 will be scattered into the cleaning solution. The second motor 53 rotates forward again, so that the magnetic rod 22 abuts against the lower end of the magnetic sleeve 212. Under the action of magnetic force, the micro magnetic beads are adsorbed to the lower end of the outer side of the magnetic sleeve 212. Then, the second motor 53 rotates reversely again, so that the magnetic rod 22 moves upward to move away from the lower end of the magnetic sleeve 212. After being subjected to the above process again, all the micro magnetic beads are scattered into the cleaning solution in the first cleaning hole 82 again. Repeating the above cleaning process several times continuously finishes the cleaning of the micro magnetic beads in the first cleaning hole 82.

Then, the second motor 53 rotates forward, so that the magnetic rod 22 abuts against the lower end of the magnetic sleeve 212. Under the action of the magnetic force, the micro magnetic beads are adsorbed to the lower end of the outer side of the magnetic sleeve 212. Then the first motor 43 rotates reversely, so that the magnetic sleeve 212 with the micro magnetic beads move upward. The reagent strip rack 7 is moved, so that a second cleaning hole 83 on the reagent strip 8 is placed just below the magnetic sleeve 212. The first motor 43 rotates forward, so that the magnetic sleeve 212 with the micro magnetic beads is immersed into the second cleaning hole 83, and the cleaning process in the first cleaning hole 82 is repeated.

The magnetic sleeve 212 with the micro magnetic beads is immersed into a third cleaning hole 84. After repeating the cleaning process in the first cleaning hole 82, the first motor 43 rotates reversely to raise the magnetic sleeve 212 adsorbed with the micro magnetic beads. The reagent strip rack 7 is moved, so that a fourth cleaning hole 85 on the reagent strip 8 is placed just below the magnetic sleeve 212. The magnetic sleeve 212 adsorbed with the micro magnetic beads is immersed into the cleaning solution in the fourth cleaning hole 85 again. The second motor 53 rotates reversely, so that the magnetic rod 22 moves away from the lower end of the magnetic sleeve 212. After cleaning for three times, the micro magnetic beads lose the magnetic attraction of the magnetic rod 22, which are scattered into the cleaning solution in the fourth cleaning hole under the action of gravity. The first motor 43 rotates reversely to drive the first plate 31, the rearing plate 3, the clamping plate 57 and the second plate 51 to move upward synchronously via the first lead screw 44. After completing the cleaning of the micro magnetic beads, the reagent strip 8 on the reagent strip rack 7 is moved for a test work of a next process.

What is provided above is the preferred embodiments according to the present application, and the protection scope of the present application is not limited to the above embodiments. Therefore, all equivalent changes made according to the structure, the shape and the principle of the present application should be considered as falling within the protection scope of the present application.

What is claimed is:

1. A cleaning device, comprising an inverted U-shaped frame body comprising a horizontal beam, wherein both ends of the horizontal beam are respectively connected with two vertical beams perpendicular to the horizontal beam; a horizontal bearing plate for bearing a cleaning mechanism and a first driving mechanism for driving the bearing plate to move up and down are provided in the frame body; the cleaning mechanism comprises a magnetic sleeve assembly and a magnetic rod in a sleeve connection with the magnetic sleeve assembly, and an upper end of the magnetic rod is connected with a second driving mechanism for driving it to move up and down relative to the magnetic sleeve assembly;
the magnetic sleeve assembly comprises a magnetic sleeve seat, a magnetic sleeve and the magnetic rod; the bearing plate is provided with multiple vertical through holes, a lower end of the through hole is fixedly communicated with the hollow tubular magnetic sleeve seat, and a lower end of the magnetic sleeve seat is in the sleeve connection with the tubular magnetic sleeve seat having an upper opening; the magnetic rod penetrates through the through hole and the magnetic sleeve seat downward respectively, and is in the sleeve connection with the magnetic sleeve;
the first driving mechanism comprises a first plate provided at a rear side of and fixedly connected with the bearing plate; a horizontal supporting plate fixedly connected with the two vertical beams is provided below the first plate; a vertically provided first motor is fixedly connected with an upper end of the horizontal beam, an output end of the first motor extends through the horizontal beam and is connected with a vertical first lead screw; the first plate is fixed connected with a first lead screw nut adapted to the first lead screw, and is further provided with a first guiding mechanism to guide it to move up and down;
the second driving mechanism comprises a second plate provided above the first plate, and the rear side of the first plate is fixedly connected with a vertical motor frame, and an upper end of the motor frame is fixedly provided with a vertical second motor; an output end of the second motor is connected with a vertical second lead screw, and the second plate is fixedly connected with a second lead screw nut adapted to the second lead screw, and is further provided with a second guiding mechanism for guiding it to move up and down; and the front side of the second plate is fixedly connected with a clamping plate for clamping the magnetic rod; and
the first motor and the second motor are connected with a PLC coordinating a movements of the first motor and the second motor.

2. The cleaning device according to claim 1, wherein the magnetic rod is rod-shaped, a lower portion of the magnetic rod is a magnetic portion, and an upper portion of the magnetic rod is provided with a retaining ring protruding outward and a top end of the magnetic rod is provided with a vertical internal threaded hole.

3. The cleaning device according to claim 2, wherein the clamping plate is provided with multiple stepped grooves with openings facing the second plate; a step at a lower end of stepped groove is provided with a clamping hole adapted to the magnetic rod; and the upper end of the magnetic rod extends through the clamping hole upward and the retaining ring abuts against the lower end of the clamping plate, and a fastener is threaded into the internal threaded hole.

4. The cleaning device according to claim 1, wherein the first guiding mechanism comprises first sliding holes provided on the first plate and at two sides of the first lead screw, and a vertical first sliding rod extends through the first sliding hole, and both ends of the first sliding rod are fixedly connected with the horizontal beam and the supporting plate respectively;
and the second guiding mechanism comprises second sliding holes provided on the second plate and at two sides of the second lead screw, and a vertical second sliding rod extends through the second sliding hole, and both ends of the second sliding rod are fixedly connected with the horizontal beam and the supporting plate respectively.

5. The cleaning device according to claim 4, wherein a first sliding sleeve is coaxially fixedly provided in the first sliding hole, and the first sliding sleeve is slidably connected with the first sliding rod; and a second sliding sleeve is coaxially fixedly provided in the second sliding hole, and the second sliding sleeve is slidably connected with the second sliding rod.

6. The cleaning device according to claim 1, wherein the other end of the first lead screw and the other end of the second lead screw are both free ends.

7. The cleaning device according to claim 4, wherein the first plate is provided with pocket holes for the second sliding rod and the second lead screw respectively; and the second plate is provided with the pocket holes for the first sliding rod and the first lead screw respectively.

8. The cleaning device according to claim 3, wherein numbers of the through holes, the cleaning mechanisms and the stepped grooves on the bearing plate are eight respectively.
